(19) United States
(12) Patent Application Publication
(10) Pub. No.: US 2012/0095052 A1
(43) Pub. Date: Apr. 19, 2012

(54) PAR-1 ANTAGONISM IN FED OR ANTACID-DOSED PATIENTS

(76) Inventors: Larisa Reyderman, Watchung, NJ (US); Teddy Kosoglou, Jamison, PA (US)

(21) Appl. No.: 13/148,799

(22) PCT Filed: Feb. 9, 2010
(86) PCT No.: PCT/US10/23628

§ 371 Date: Jan. 6, 2012

Related U.S. Application Data
(60) Provisional application No. 61/152,080, filed on Feb. 12, 2009.

(51) Int. Cl. A61K 31/443; A61P 7/02; A61P 9/00

(52) U.S. Cl. 514/337

(57) ABSTRACT

Disclosed are methods of inhibiting a PAR-1 receptor in a patient who has recently ingested food or an antacid comprising the step of administering an effective amount

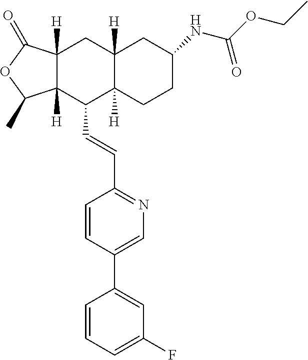

and/or the bisulfate salt thereof.

### BACKGROUND OF THE INVENTION

PAR-1 antagonists are thought to have a variety of therapeutic uses, including, inter alia, those related to the treatment or prevention of cardiovascular, inflammatory, and proliferative conditions. A sampling of the literature regarding the potential uses of PAR-1 antagonists is found in U.S. Ser. No. 10/705,282.

It is known that the recent ingestion of food by a patient can negatively impact on efficacy a of drug substance subsequently administered to that patient. This effect can be due to a variety of mechanisms, such as delayed bioabsorption and/or impaired metabolism. The recent ingestion of an antacid is also thought to be a potential risk to efficacy. Patients who are at risk for or who suffer from the above-described conditions and who are administered a PAR-1 antagonist are subject to these same potential threats to efficacy.

The compound of the following structural formula:

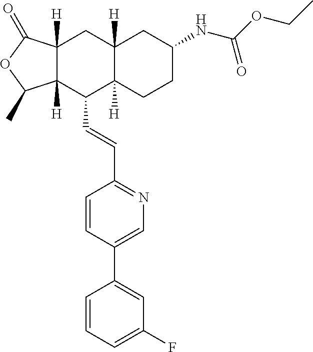

and pharmaceutically acceptable salts thereof, including its bisulfate salt form, is a PAR-1 antagonist currently in development for the treatment of acute coronary syndrome and secondary prevention of cardiovascular events. The chemical name of this compound is ethyl [(R1R,3aR,4aR,6R,8aR,9S,9aS)-9-[(E)-2-[5-(3-fluorophenyl)-2- pyridinyl]ethenyl]-dodecahydro-1-methyl-3-oxonaphtho[2,3-c]furan-6-yl] carbamate. It has been disclosed in U.S. Pat. No. 7,304,078, crystalline forms of the bisulfate salt are disclosed in U.S. Pat. No. 7,235,567, formulations are disclosed in Ser. Nos. 11/771,571; 11/960,320; 11/771,520; and 11/860,165; and methods of treating a variety conditions are disclosed in Ser. Nos. 10/705,282; 60/753,246; 11/642,505; and 11/642,487, all of which are herein incorporated in their entirety. It would be a significant improvement in the treatment of the relevant disease states to identify a PAR-1 antagonist whose efficacy after recent ingestion of food or an antacid was superior to the post-ingestion efficacy of competitive other treatment options.

### DESCRIPTION OF THE FIGURES

FIG. 1 displays the mean plasma concentration of SCH 530348, which represents

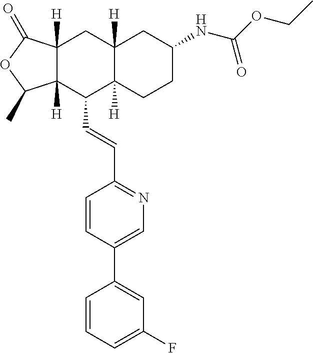

and/or its bisulfate salt form in fasted and fed subjects.

FIG. 2 displays the mean plasma concentration of SCH 530348 (also known as TRA), which represents

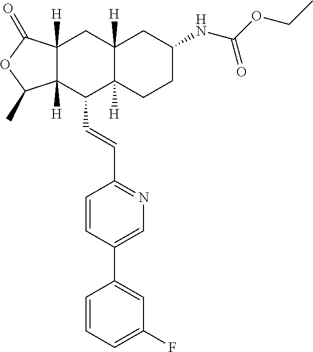

and/or the bisulfate salt form thereof, in fasted subjects with and without an antacid.

### DETAILED DESCRIPTION

The effect of food And antacid on the pharmacokinetics of

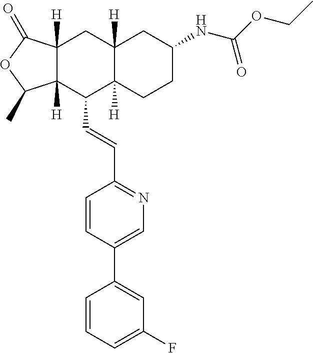

and or the bisulfate salt thereof in healthy subjects was studied in a clinical trial. The primary objective of the study was to evaluate the effect of food (standardized high-fat breakfast) and antacid (increased gastric pH) on the pharmacokinetics (PK) of

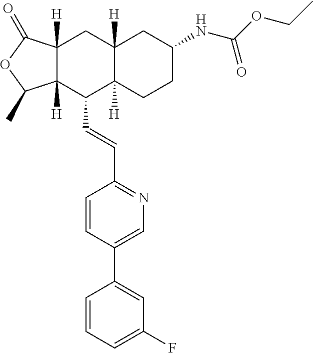

and or the bisulfate salt thereof administered orally as a 40 mg tablet. A secondary objective was to evaluate the effect of meal timing relative to fasting on the PK of

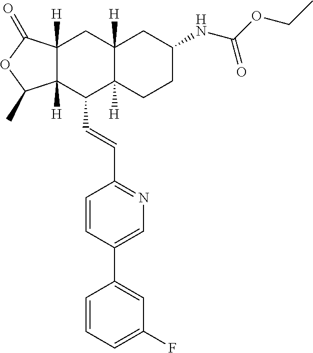

and/or the bisulfate salt thereof administered orally as a 40 mg tablet.

The study was designed as a randomized, open-label, single-dose, parallel-group, single-center study conducted in healthy young adults in conformance with Good Clinical Practice. Healthy male and female subjects between the ages of 18 and 45 years with a body mass index of 19-32 kg/m² were eligible for enrollment. Eligible subjects were randomly assigned to receive a single 40 mg dose of

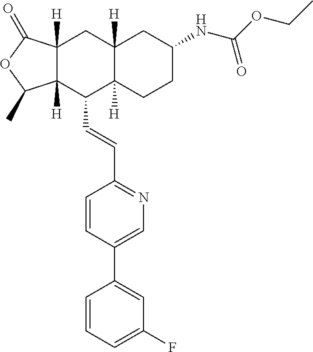

and/or the bisulfate salt thereof as follows:

| | |
|---|---|
| Group A | Fasted (after a 10-hour fast) |
| (n = 22) | |
| Group B | Fed (0 hours; within 5 minutes of completing a |
| (n = 20) | standardized high-fat breakfast) |
| Group C | 1 hour after completing a standardized high-fat |
| (n = 11) | breakfast |
| Group D | 2 hours after completing a standardized high-fat |
| (n = 10) | breakfast |
| Group E | Antacid (after a 10-hour fast and within 5 minutes after |
| (n = 20) | drinking 20 mL Gaviscon ® extra-strength liquid antacid) |

Baseline demographic characteristics are summarized in Table 1.

TABLE 1

| | Fasted | | Fed | | 1 hr after Food | | 2 hrs after Food | | Antacid | |
|---|---|---|---|---|---|---|---|---|---|---|
| Characteristic | (n = 22) | | (n = 20) | | (n = 11) | | (n = 10) | | (n = 20) | |
| Sex (n, %) | | | | | | | | | | |
| Female | 9 | (41) | 9 | (45) | 7 | (64) | 4 | (40) | 9 | (45) |
| Male | 13 | (59) | 11 | (55) | 4 | (36) | 6 | (60) | 11 | (55) |
| Race (n, %) | | | | | | | | | | |
| White | 17 | (77) | 10 | (50) | 6 | (55) | 5 | (50) | 14 | (70) |
| Non-white | 5 | (23) | 10 | (50) | 5 | (45) | 5 | (50) | 6 | (30) |
| Age | | | | | | | | | | |
| Mean (SD) | 30.0 | (8.4) | 26.3 | (7.9) | 27.1 | (8.3) | 28.4 | (6.7) | 26.6 | (6.6) |
| Median | 27.5 | | 23.5 | | 25.0 | | 27.0 | | 24.5 | |
| Range | 20-43 | | 18-43 | | 19-42 | | 20-41 | | 19-41 | |
| BMI (kg/m²) | | | | | | | | | | |
| Mean | 26.99 | (2.91) | 23.10 | (2.63) | 23.23 | (2.88) | 24.64 | (2.70) | 24.11 | (3.30) |

| | | | | | |
|---|---|---|---|---|---|
| Median | 27.25 | 22.15 | 23.00 | 24.60 | 23.45 |
| Range | 22.6-32.0 | 19.7-27.4 | 19.6-28.2 | 19.0-28.0 | 19.1-29.5 |

SD = standard deviation
Blood samples were collected at predose (0 hour) and at 0.5, 1, 1.5, 2, 3, 4, 6, 12 and 24 hours after dosing.
Additional blood samples were collected on an outpatient

basis on Days 7, 14, 21, 28, 35, and 42 (±1 day) after dosing. The bioanalytical assay was an LC-MS/MS assay, LLOQ=1.00 ng/mL.

The study results demonstrate the following effects of food and antacid on pharmacokinetics of

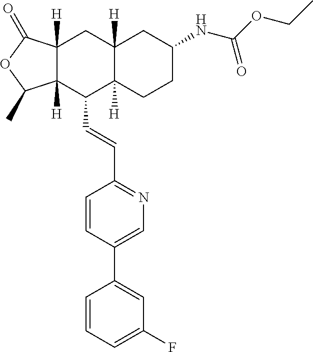

and/or the bisulfate salt thereof. The compound represented by the chemical structure

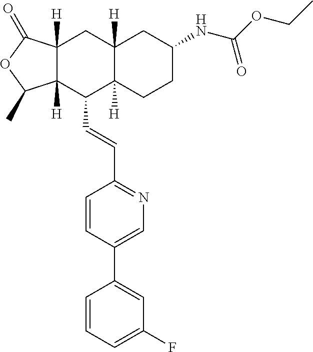

and/or the bisulfate salt thereof, was rapidly absorbed in fasted subjects (median Tₘₐₓ: 1 hour) , whereas its absorption was delayed in fed subjects: median Tₘₐₓ was 3 hours when

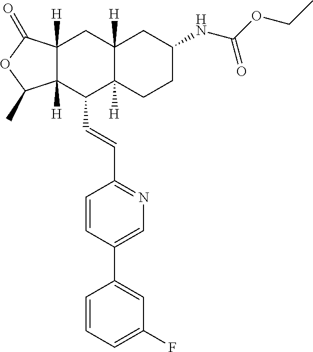

and/ or the bisulfate salt thereof was administered immediately with food, and 2 hours when administered 1 hour or 2 hours after a meal (FIG. 1 and Table 2). Co-administration of

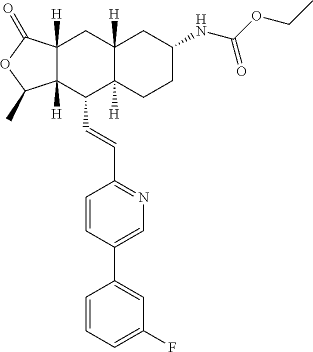

and/or the bisulfate salt thereof with antacid in fasted subjects was associated with delayed absorption (median Tₘₐₓ: 2 hours vs 1 hour in fasted subjects without antacid; Table 2 and FIG. 2):

Table 2 displays mean (% coefficient of variation) pharmacokine values after single 40 mg dose of

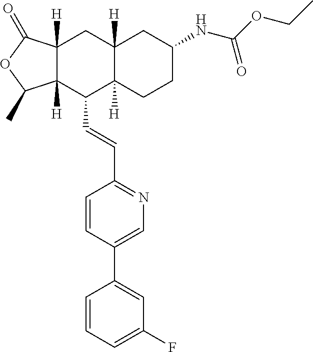

and/or the bisulfate salt in fasted and fed subjects.

TABLE 2

| Treat- | | | AUC(tf) | AUC(I) | |
|---|---|---|---|---|---|
| ment | Cₘₐₓ | Tₘₐₓ^{a} | (ng · hr/ | (ng · hr/ | |
| Group | (ng/mL) | (hr) | mL) | mL) | t_{1/2} (hr) |
| Fasted | 262 | 1 | 16800 | 18400^{b} | 240^{b} |
| without | (33) | (0.42-3) | (23) | (n = 21) | (41) |
| antacid | | | | | |
| (n = 21) | | | | | |
| Fed | 347 | 3 | 22800 | 26600^{c} | 261^{c} |
| (n = 20) | (43) | (1-4) | (27) | (29) | (57) |
| 1 hour | 298 | 2 | 23200 | 26800^{d} | 289^{d} |
| after | (24) | (1.5-4) | (33) | (34) | (44) |
| food | | | | | |
| (n = 11) | | | | | |
| 2 hours | 355 | 2 | 22400 | 23400^{d} | 242^{d} |
| after | (37) | (1-4) | (25) | (23) | (34) |
| food | | | | | |
| (n = 10) | | | | | |
| Fasted | 165 | 2 | 15000 | 15800^{c} | 245^{c} |
| with | (40) | (1-6) | (29) | (29) | (42) |
| antacid | | | | | |
| (n = 20) | | | | | |

^{a}Median (range)
^{b}n = 19
^{c}n = 18
^{d}n = 9.

FIG. 2. Mean plasma concentration of

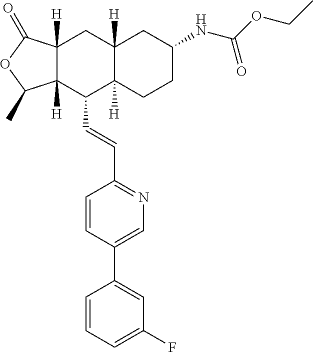

and/or the bisulfate salt thereof in fasted subjects without and with antacid.

The data demonstrate that administration of

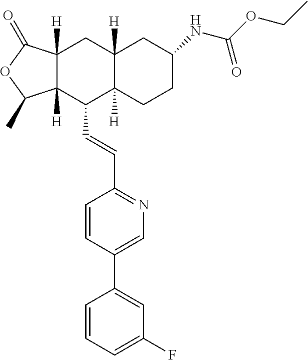

and/or the bisulfate salt thereof 40 mg tablet immediately with food increased systemic exposure (AUC[l]) by 43% and peak plasma concentration (Cₘₐₓ) by 31% (Table 3); similar increases were observed when the tablet was administered 1 hour or 2 hours after a meal (Table 3). Table 3 displays the relative bioavailability of

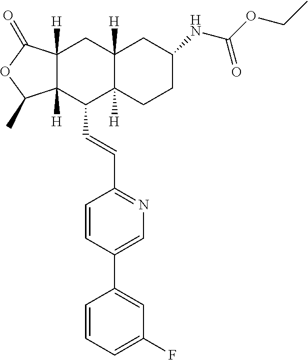

and/or the bisulfate salt thereof in fasted and fed subjects

TABLE 3

| | | | | 90% |
|---|---|---|---|---|
| | Treatment | | Ratio Estimate | Confidence |
| Parameter | Group | n | Fed vs Fasted | Interval |
| AUC(I)^{a} | Fasted | 19 | — | — |
| | Fed | 18 | 143 | 123-166 |
| | 1 hr after food | 9 | 143 | 119-172 |
| | 2 hr after food | 9 | 127 | 106-153 |
| Cₘₐₓ | Fasted | 21 | — | — |
| | Fed | 20 | 131 | 108-159 |
| | 1 hr after food | 11 | 117 | 93-147 |
| | 2 hr after food | 10 | 137 | 107-174 |

^{a}AUC(I) could not be determined for some subjects

The data show that antacid co-administration reduced the systemic exposure (AUC[l]) of

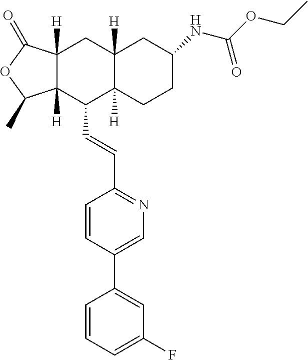

and/or the bisulfate salt thereof by 15% and its peak plasma concentration (Cₘₐₓ) by 38% (Table 4). Table 4 shows the relative bioavailability of

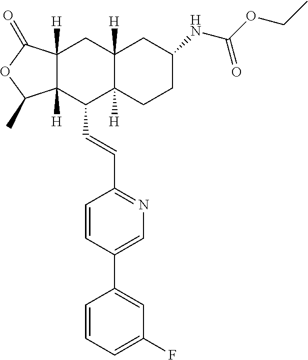

and/or the bisulfate salt thereof in fasted subjects administered alone and with antacid.

TABLE 4

| | | | Ratio Estimate | 90% |
|---|---|---|---|---|
| | | | with vs without | Confidence |
| Parameter | Treatment Group | n | Antacid | Interval |
| AUC(I)^{a} | 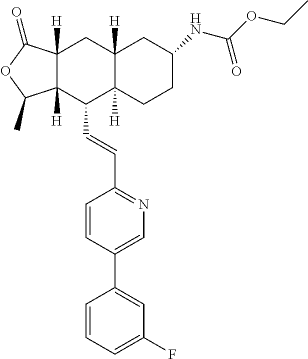 and/or the bisulfate salt thereof alone With antacid | 19 18 | 85 | 73-99 |
| Cₘₐₓ | 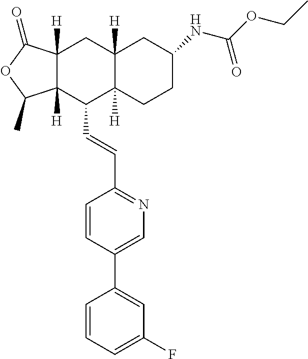 and/or the bisulfate salt thereof alone With antacid | 21 20 | 62 | 51-75 |

^{a}AUC(I) could not be determined for some subjects.

The result of this study support the following conclusions:
Food delayed the absorption of

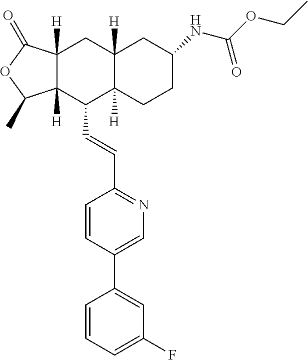

and/or the bisulfate salt thereof administered as a 40 mg oral tablet and increased the peak plasma concentration and exposure to

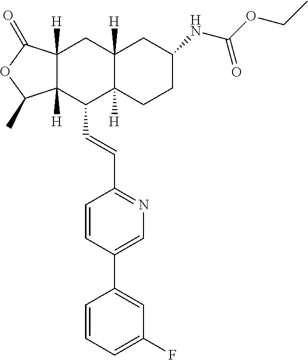

and/or the bisulfate salt thereof. The effect on exposure was similar irrespective of whether it was administered immediately after or 1 or 2 hours after a meal,
Co-administration of the

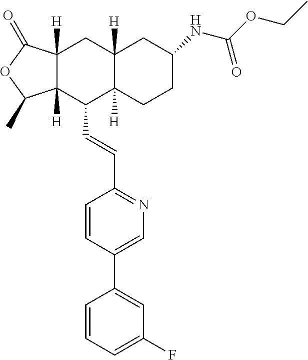

and/ or the bisulfate salt thereof 40 mg oral tablet with antacid resulted in the delay absorption and decreased peak plasma concentration and exposure to

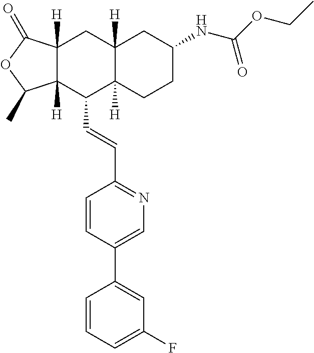

and/or the bisulfate salt thereof.
The effects of concomitant food and antacid on the pharmacokinetics of

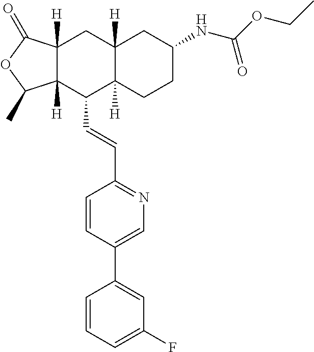

and/or the bisulfate salt thereof are modest and not considered to be of clinical significance during chronic treatment, indicating that

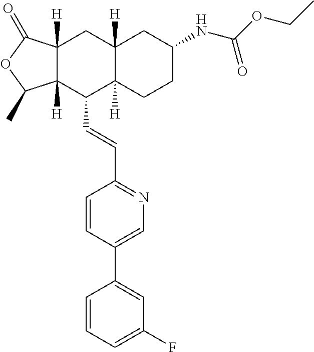

and/or the bisulfate salt thereof can be safely co-administered with food and antacids. In situations where a loading dose of

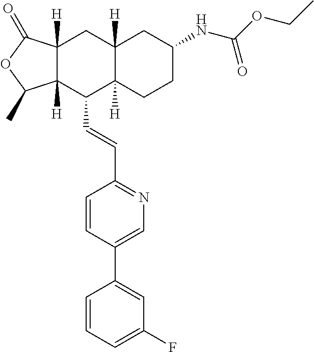

and/or the bisulfate salt thereof may be required, such as in patients with an acute coronary syndrome, concomitant food and/or antacid may slightly delay the onset of action, but should have no significant effect on the overall extent of platelet aggregation inhibition.

Thus, it appears that

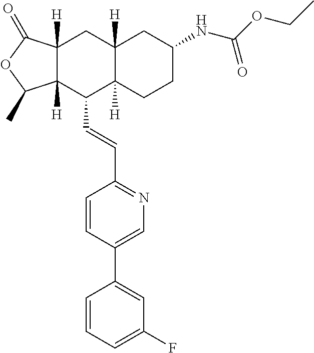

and/or the bisulfate salt thereof is effective in treating fed patients and those who have taken an antacid for those conditions amenable to treatment by a PAR-1 inhibitor, e.g., acute coronary syndrome and peripheral arterial disease. Secondary prevention of coronary events can also be effected in such patients by administration of

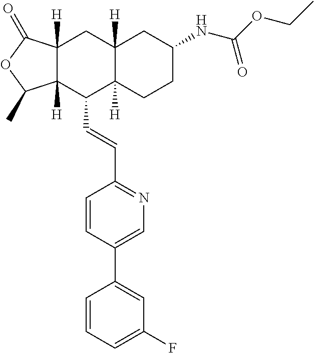

and/or the bisulfate salt thereof.

The pharmacokinetic characteristics of

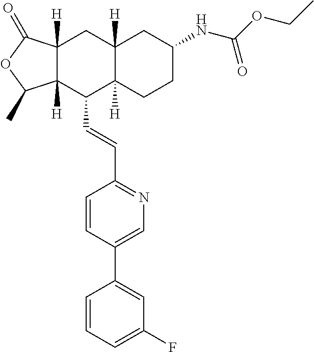

and/or the bisulfate salt thereof demonstrated in this study (e.g., Cₘₐₓ, Tₘₐₓ and AUC) may be superior to those of other anticoagulants in patients who have recently ingested food or an antacid.

Materials summarizing this study are presented in Appendix I of this specification. Materials summarizing an earlier study that also looked at the effect of food on the oral bioavalibility of

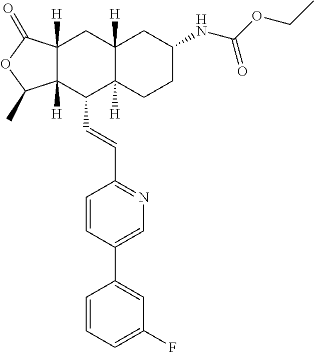

and/or the bisulfate salt thereof are presented as Appendix II of this specification.

The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

We claim:

1. A method of inhibiting a PAR-1 receptor in a patient in need of such inhibition and who has recently ingested food or an antacid comprising the step of administering an effective amount of 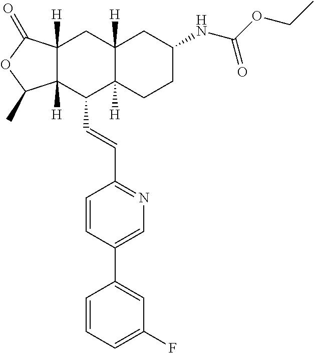
and/or the bisulfate salt thereof.

2. The method according to claim 1 wherein said patient has ingested food within 2 hours of said administration of 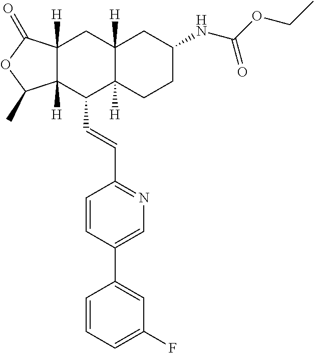
and/or the bisulfate salt thereof.

3. The method according to claim I wherein said patient has ingested food within 1 hour of said administration of 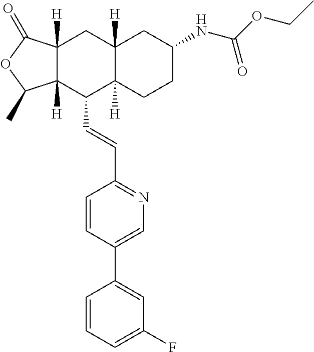
and/or the bisulfate salt thereof.

4. The method according to claim I wherein said patient has ingested food or an antacid within 5 minutes of said administration of 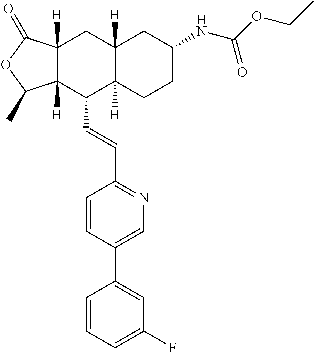
and/or the bisulfate salt thereof.

5. A method of treating a cardiovascular condition in a patient in need of such treatment comprising the step of administering to the patient an effective amount of 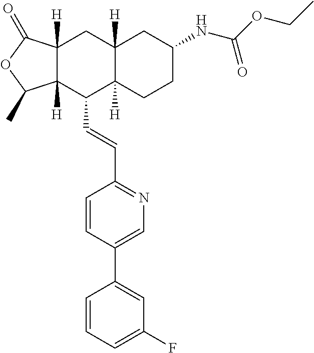
and/or the bisulfate salt thereof within 2 hours of said patient having ingested food or an antacid.

6. The method according to claim 5, wherein said cardiovascular condition is acute coronary syndrome.

7. The method according to claim 5, wherein said cardiovascular condition is peripheral arterial disease.

8. The method according to claim 5, wherein said effective amount of 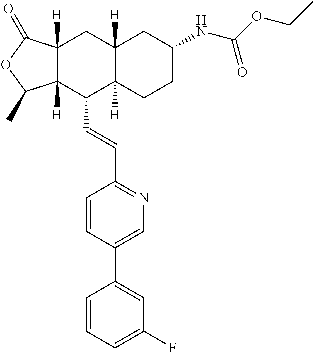
and/or the bisulfate salt thereof is about 40 mg.

9. A method of effecting secondary prevention of a cardiovascular event in a patient in need of such prevention comprising the step of administering to the patient an effective amount of 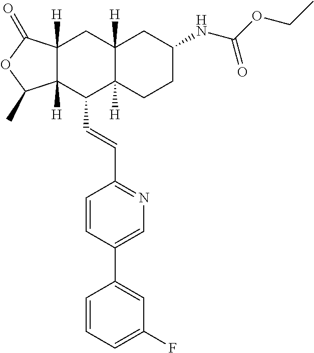
and/or the bisulfate salt thereof within 2 hours of said patient having ingested food or an antacid.

10. A method of obtaining a maximum mean plasma concentration of an anticoagulant (Cₘₐₓ) at a time (Tₘₐₓ) of not more than 4 hours after administration of the anticoagulant to the patient who ingested food within 2 hours of said administration.

11. A method of obtaining a maximum mean plasma concentration of an anticoagulant (Cmax) at a time (Tmax) of not more than 6 hours after administration of the anticoagulant to the patient who ingested an antacid within 5 minutes of said administration.
